(19) United States
(12) Patent Application Publication
(10) Pub. No.: US 2010/0057025 A1
(43) Pub. Date: Mar. 4, 2010

(54) Device for Treating Wounds

(75) Inventor: Martin Aicher, Zurich (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MEDELA HOLDING AG, Baar (CH)

(21) Appl. No.: 12/514,452

(22) PCT Filed: Nov. 9, 2007
(86) PCT No.: PCT/CH07/00551

§ 371 Date: May 11, 2009

(30) Foreign Application Priority Data
Nov. 30, 2006 (CH) 1943/06

(51) Int. Cl. A61F 13/00; A61M 1/00

(52) U.S. Cl. 604/319; 602/43

(57) ABSTRACT

The invention relates to a device for treating wounds of a patient by means of low pressure. Said device comprises a covering for forming a low pressure chamber over the wound. Said covering is secured to the skin surrounding the wound. The shape of the covering can be modified from a base shape into a predefined shape for use after applying to the wound and securing to the skin. Preferably, the covering comprises a shape memory.

### TECHNICAL FIELD

The invention relates to a device for treating wounds according to the preamble of claims 1 and 23.

### PRIOR ART

Open wounds that are too large or too badly inflamed to heal independently have long been a problem in medicine. It has been found that wound treatment using low pressure stimulates, supports and accelerates the healing of the wound. This type of treatment is known as wound drainage.

U.S. Pat. No. 5,636,643, for example, discloses a wound drainage device with a fluid-impermeable and gas-impermeable rigid cover that is placed over a wound and is secured to the healthy skin outside the margins of the wound. Underneath the cover, a foam is placed onto or into the wound. A negative pressure is generated in the cover from outside by means of a vacuum pump in order to accelerate the healing of the wound.

WO 03/018098 also describes a device for treating wounds, with a cover and with a porous pad that is placed onto the wound underneath the cover. This document proposes automated oscillation of the negative pressure in order to stimulate the healing of the wound.

WO 2006/056408 proposes that the cover be provided with supply devices for treatment substances. These treatment substances are removed together with the wound secretions through a drainage device.

WO 2006/048246 discloses a multicomponent dressing for wound treatment by means of negative pressure. This dressing comprises superabsorbent polymers, wherein the absorbed wound secretions remain bound to polymers in the wound cavity.

In US 2006/0155260, the wound is cleansed with a fluid and a closed circuit is used.

WO 2004/071279 describes a wound treatment device that comprises sensors for monitoring the healing of the wound.

WO 2006/081221 further proposes the treatment of wounds by phototherapy.

### DISCLOSURE OF THE INVENTION

It is therefore an object of the invention to create a device for treating wounds that can be used for different sizes of wounds.

This object is achieved by a device for treating wounds that has the features of claim 1.

The device according to the invention for treating wounds of a patient, preferably by means of low pressure, comprises a covering for forming a low-pressure chamber over the wound, wherein the covering can be secured on the skin surrounding the wound. After the covering has been applied to the wound and secured on the skin, the shape of the covering can change from a basic shape to a predefined shape for use.

This wound covering can easily be adapted specifically to the corresponding wound. It avoids a mechanical pressure on the wound bed but still permits a contraction of the wound. It is also relatively simple to use, such that application to the wound and removal from the wound take little time.

The covering can preferably change automatically from the basic shape to the shape for use. This has the advantage that touching the wound, through incorrect use of the covering, can be largely avoided.

The covering is preferably made at least partially, preferably entirely, from a material with shape memory. The covering preferably has the shape for use at a temperature that corresponds approximately to a body temperature of the patient. The basic shape is preferably present at room temperature (i.e. about 20° C.). All suitable materials with shape memory can be used, in particular polymers or metals.

Additionally or alternatively, however, the covering can also be provided with at least one mechanical element by means of which its shape can be changed. This element can be a restoring spring, for example.

In one embodiment, the covering comprises a base element without shape memory and at least one subcomponent with shape memory that is arranged on or in this base element, wherein the at least one subcomponent extends over the base element in such a way that, upon changing shape, it forces the base element to undergo a predetermined change of shape.

In another embodiment, the covering comprises a profile element, which can be bent to form a ring, and a cover sheeting or film that can be stretched over the ring. The profile element can preferably change its profile height in a controlled manner mechanically, e.g. by a spring, by shape memory, by being inflated, or by virtue of its coefficient of thermal expansion.

One or more of the following means can be arranged in the covering: an ultrasound transmitter, a light source, a temperature sensor, a sensor for measuring the humidity, a sensor for measuring the flow of blood, a sensor for measuring the bacterial colonization, a heating means.

The device according to the invention is preferably used for wound drainage by low pressure. However, it can also be used for other wound treatments. Moreover, a wound is understood here not only as an open area of skin but also as skin anomalies or other tissue defects.

The system according to the invention for treating wounds comprises a device of the abovementioned type for treating wounds and also a vacuum pump that can be connected to said device so as to generate a low pressure in a cavity generated by the device for treating wounds.

In the method according to the invention for treating wounds, a covering is secured on the wound, wherein the covering has a basic shape when being secured, and wherein the covering, after it has been secured in place, is changed to a shape for use.

Other advantageous embodiments and variants of the method are set forth in the dependent claims.

### BRIEF DESCRIPTION OF THE DRAWINGS

The subject matter of the invention is explained below on the basis of preferred illustrative embodiments depicted in the attached drawings, in which:

FIG. 1 shows a cross section through a covering according to the invention, in a basic shape prior to its use, according to a first embodiment;

FIG. 2a shows a cross section through the covering according to FIG. 1 in a basic shape;

FIG. 2b shows a perspective view of the covering according to FIG. 2a;

FIG. 3a shows a cross section through a wound and surrounding tissue;

FIG. 3b shows a perspective view of the wound according to FIG. 3a;

FIG. 4a shows a cross section through the wound according to FIG. 3a, with the covering according to FIG. 2a placed over it;

FIG. 4b shows a perspective view of the wound and covering according to FIG. 4a;

FIG. 5a shows a cross section through the wound and through the drainage device secured over the latter in a basic shape;

FIG. 5b shows a perspective view of the wound and the device according to FIG. 5a;

FIG. 6 shows a cross section through the wound and the device according to FIG. 5a in a shape for use;

FIG. 7a shows a graphic representation of the applied vacuum as a function of time;

FIG. 7b shows a graphic representation of a hydrogel application as a function of time;

FIG. 7c shows a graphic representation of an irrigation as a function of time;

FIG. 8a shows a cross section through the wound and drainage device in a hydrogel application in a first state and

FIG. 8b in a second state;

FIG. 9 shows a cross section through the drainage device according to the invention in a second embodiment;

FIG. 10 shows a cross section through the drainage device according to the invention in a third embodiment;

FIG. 11a shows a cross section through the drainage device according to the invention in a fourth embodiment;

FIG. 11b shows a graphic representation of the measurement of the flow of blood;

FIG. 12 shows a cross section through the drainage device according to the invention in a fifth embodiment;

FIG. 13a shows a perspective view of a sixth embodiment of the drainage device according to the invention in a basic shape and

FIG. 13b in a shape for use;

FIG. 13c shows a cross section through the device according to FIG. 13b;

FIG. 14 shows a view of a first variant of a subcomponent according to the invention;

FIG. 15 shows a view of a second variant of a subcomponent according to the invention;

FIG. 16a shows a perspective view of a profile element of a drainage device according to a seventh embodiment;

FIG. 16b shows the profile element according to FIG. 16a in an arrangement around a wound, and

FIG. 16c shows a cross section through the drainage device according to the seventh embodiment.

### WAYS OF CARRYING OUT THE INVENTION

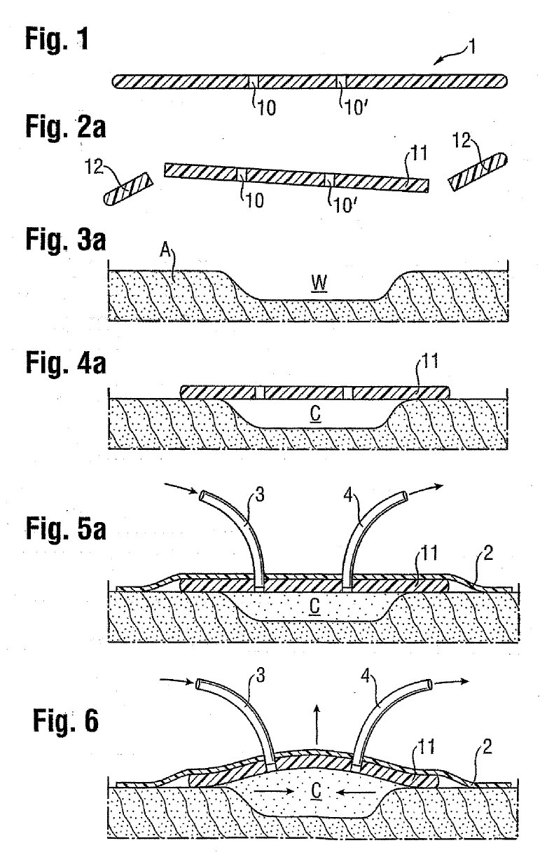

FIGS. 1 to 6 show a first preferred embodiment of the device according to the invention for treating wounds, in particular a wound drainage device. The starting material is a cover plate 1 according to FIG. 1, which preferably has a plane configuration, particularly a plane-parallel configuration. This plate 1 has at least two, and possibly also three, four or more through-openings 10, 10′ extending preferably at right angles to the plate 1. However, they can also extend at an angle not orthogonal to the plate 1.

The plate 1 has a shape memory that can preferably be thermally activated. A predefined or preprogrammed shape is preferably adopted at human body temperature.

The material is preferably an SMP, i.e. a shape memory polymer, for example a block copolymer, e.g. a wax-elastomer composite. The plate is preferably rigid or at least self-supporting, such that its shape changes only under the effect of an external force and in particular such that it maintains its basic shape, and also its shape for use described below, without being supported across its entire surface area. The shape for use should in particular also be maintained in the event of a pressure difference on the two opposite sides. The plate 1 preferably has a thickness of 1 to 6 mm. In another embodiment, the shape memory polymer can be stimulated by magnetism or light. This applies also to other embodiments of the invention, in particular to the embodiments described further below.

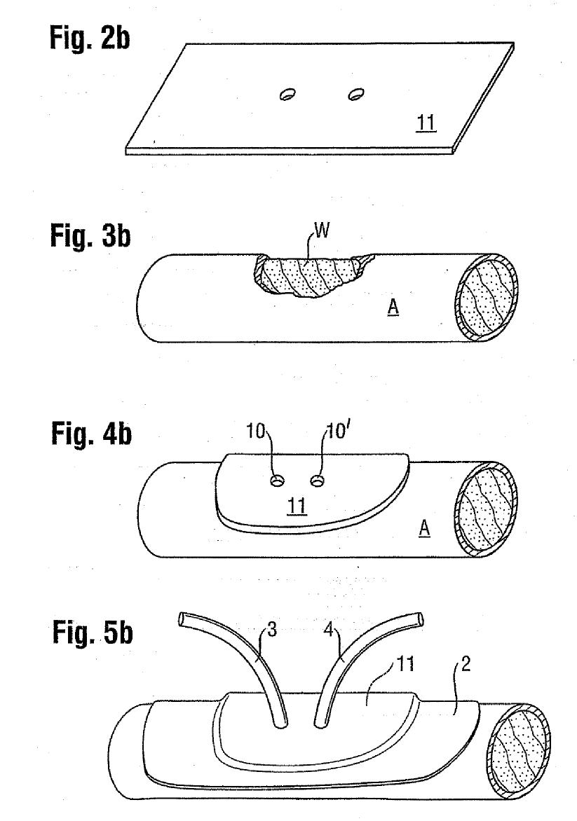

This cover plate 1 is now adapted for use to the size of the wound. A corresponding wound W is shown in FIGS. 3a and 3b. According to FIGS. 2a and 2b, the plate 1 is now accordingly cut to size, with a desired edge width 12 simply being cut off or broken off at two opposite ends. A cover plate blank 11 with the two through-openings 10, 10′ remains. This blank 11 is now placed over the wound W according to FIGS. 4a and 4b such that a cavity C is formed. According to FIGS. 5a and 5b, the blank 11 is fixed in position by means of an adhesive sheeting or film 2 or another suitable fixing element. For this purpose, the edges of the adhesive film 2 are affixed to healthy skin or tissue A surrounding the wound W. The film 2 can also adhere to the blank 11. However, it can also simply be stretched over the latter without being connected to the film.

Moreover, a delivery tube 3 is inserted into the first through-opening 10 and a drainage tube 4 is inserted into the second through-opening 10′. The tubes 3, 4 can also be fixed by means of the film 2 and/or they can be fixed in position by fixing rings, clips, separate adhesives or other suitable means.

As can be seen from FIGS. 5a and 5b, the blank 11 still has a basic shape when applied, here as a plane-parallel plate. The basic shape could also be another shape, for example it could curve inward or outward relative to the cavity C, and it could also be undulating or pyramid-shaped.

As can be seen now from FIG. 6, the blank 11 adopts a shape for use as soon as it has reached the temperature of the surrounding tissue A or of the wound W. This shape for use is preferably hood-shaped or dome-shaped, directed outward away from the wound, as is indicated by the vertical arrow. The height of the cavity C and therefore the distance of the blank 11 from the wound surface are thus increased. Mechanical pressure on the wound bed and adherence of the covering are avoided. Another important point is that the change of shape causes a contraction of the wound, since the wound margins are pulled inward, as is indicated by the horizontal arrows, and the blank 11, which is also rigid in the position of use, ensures that a constant traction or pressure is applied to the wound margins throughout the entire drainage procedure or wound treatment.

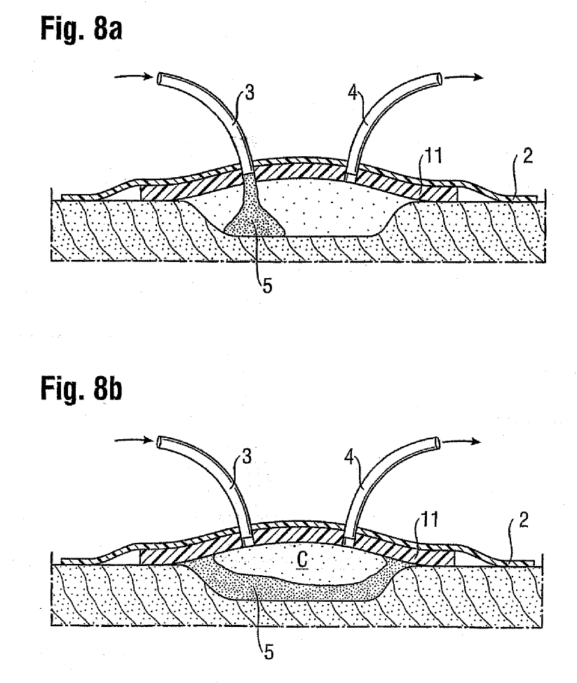

If not already begun, the wound drainage can now be started, i.e. a suitable low pressure can be applied via the drainage tube 4. Wound secretions can also be removed via the drainage tube 4. Moreover, means for accelerating the healing and disinfection of the wound can be supplied via the delivery tube 3, for example a hydrogel 5, as is shown in FIGS. 8a and 8b. The wound can also be irrigated, for example with Ringer's solution.

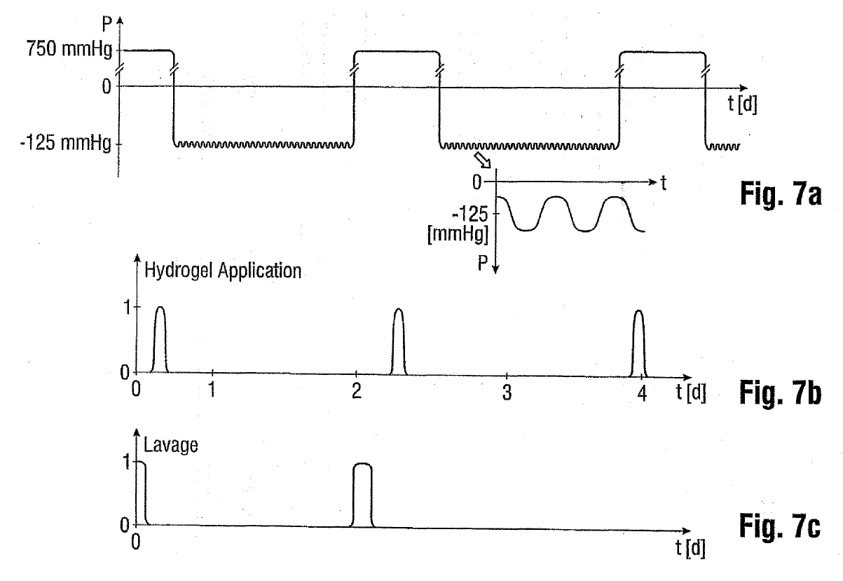

FIGS. 7a to 7c show a possible relationship between a vacuum application in the cavity C (FIG. 7a), a hydrogel application (FIG. 7b) and an irrigation of the cavity (FIG. 7c). The three time axes t are identical and an increment in each case designates 1 day d. As can be seen from FIG. 7a, a pulsing sinusoidal vacuum is applied over a long period of time. According to the figure, it is about 125 mmHg, preferably between 80 and 140 mmHg. The hydrogel application is carried out over a much shorter period than one day, preferably for 1 to 3 minutes in the space of 1 to 2 days until the wound is wet. The same applies to the irrigation, which is preferably done before the wetting of the wound bed with hydrogel and the application is not started until 0.5 to 2 hours after the irrigation. Oxygenation can take place at the same time as the application of the low pressure or as an independent phase between the applications.

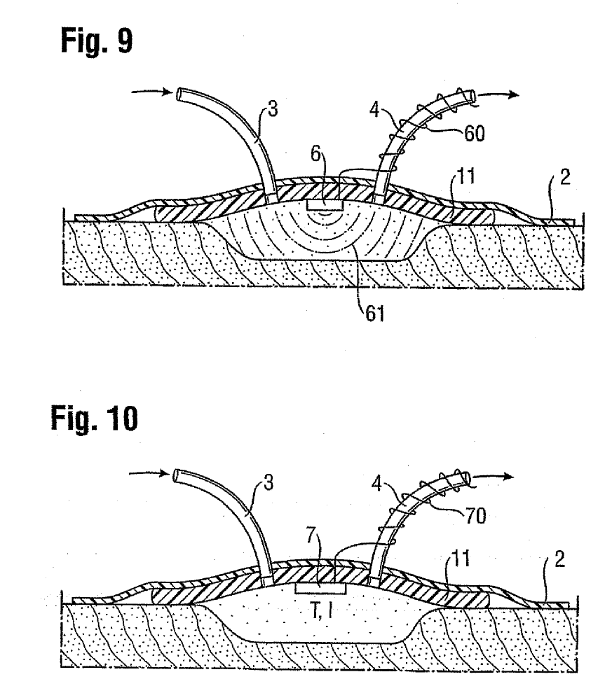

Moreover, the wound drainage device or the covering used for this can be provided with additional functions. Thus, as is shown in FIG. 9, an ultrasound transmitter 6 can be connected to the blank 11 and transmits ultrasonic waves 61 into the cavity C. This transmitter 6 can already be fitted in place by the manufacturer and supplied as a component part of the cover plate 1. However, it can also be secured in place later on. For this purpose, the cover plate 1 can have a corresponding recess or indentation. The transmitter 6 can be secured by adhesive, clips, welding, casting or other suitable means. The ultrasound transmitter 6 is preferably operated by the same device that also contains the vacuum source. In this case, it is advantageous if the line 60 for the ultrasound transmitter 6 is routed along the drainage tube 4.

Instead of the ultrasound transmitter 6, a light source can also be present to transmit light into the cavity C. The set-up corresponds to that of FIG. 9 and the above description also applies to the light source. Ultrasound transmitter and light source can also be used in combination in the wound drainage device. Both in their own way stimulate the healing of the wound. They can be used together with or instead of the vacuum.

In addition of or instead of this transmitter or this source, means for monitoring the healing of the wound can also be connected to the blank 11. The details given above relating to the time and nature of the securing also apply here. One such means is, for example, a temperature sensor and/or a hydrosensor 7, which measures the moisture in the wound. This is illustrated in FIG. 10. Here too, the signal line 70 is preferably routed along the drainage tube 4 and the data are evaluated and exploited for further treatment in the device of the vacuum pump.

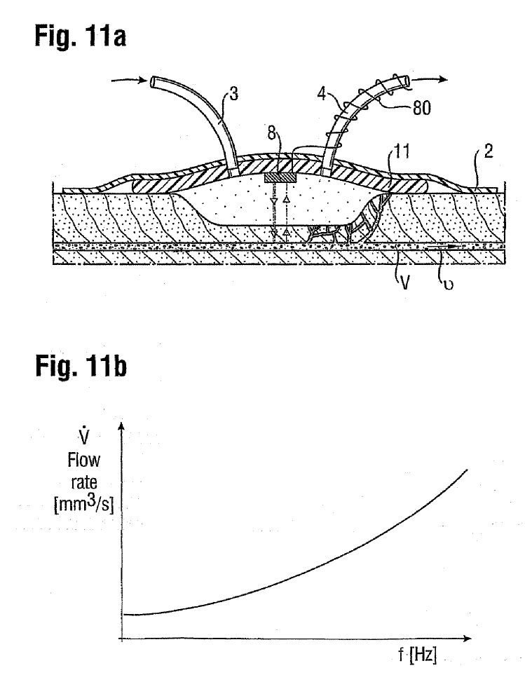

A further monitoring means is a flow sensor 8 according to FIG. 11a, which qualitatively measures the flow of blood through the tissue A lying beneath the wound and forwards the data via the signal line 80. FIG. 11b shows an example of a flow rate of the kind that can be detected by the abovementioned sensor.

It is also possible to use other monitoring sensors, however, for example a sensor for monitoring the bacterial colonization.

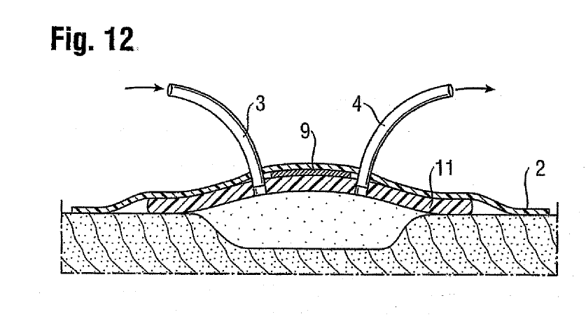

Moreover, the device can be provided additionally or alternatively with a heating element 9, in order to maintain the desired temperature. This temperature can be the optimum temperature for achieving the desired shape memory of the blank 11. However, it can also be the optimum temperature for healing of the wound. This is illustrated in FIG. 12. A resistance heating element of a known type, preferably in a flat configuration, is suitable in particular as heating element 9. However, it is also possible to use a heating element that can be activated by way of a chemical reaction or another suitable heating element. Here, the heating element 9 is arranged outside the cavity C, on the side of the blank 11 directed away from the latter, and is located under the adhesive film 2. However, it can also be arranged in the cavity C or over the adhesive film 2. If suitable, the sensors and sources described above can also be arranged outside the cavity C.

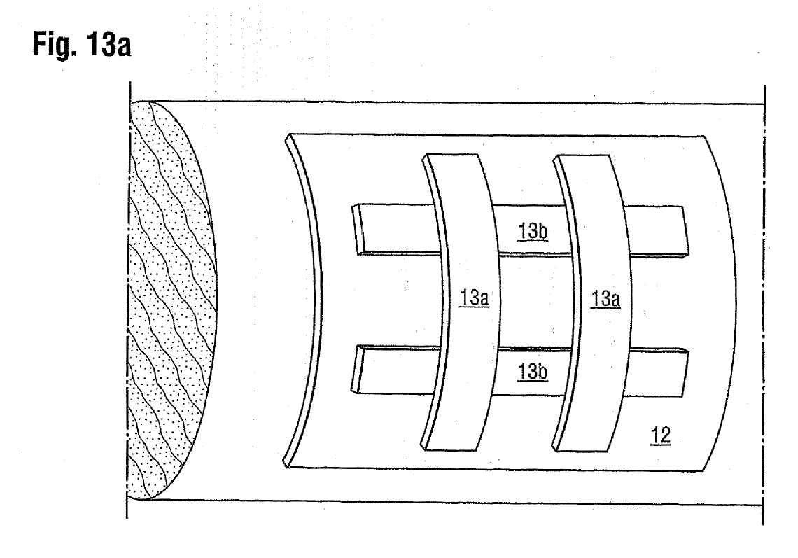

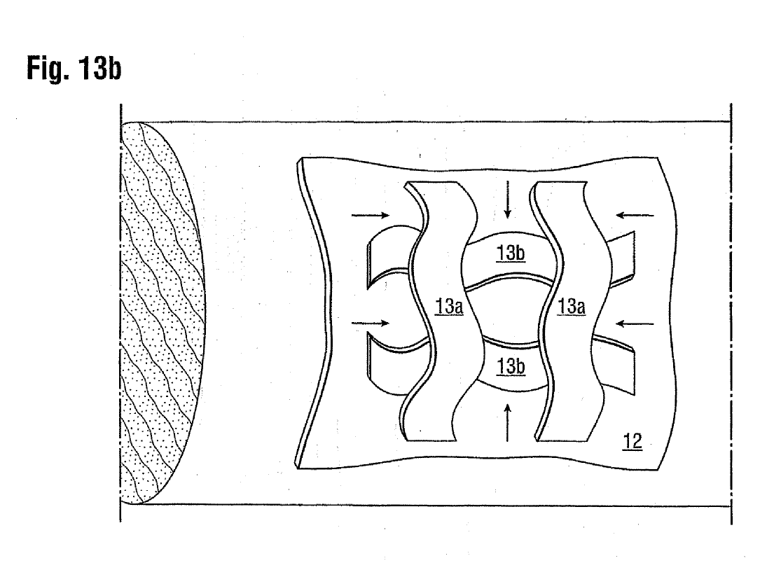

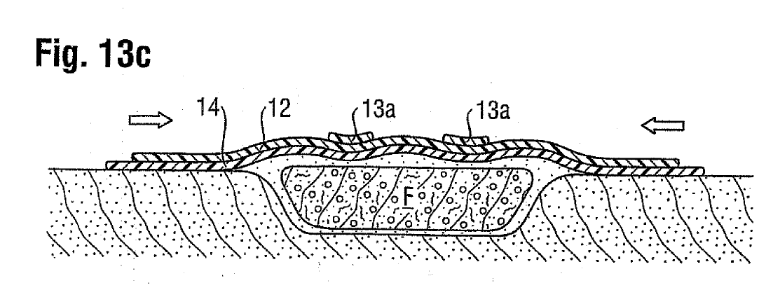

FIGS. 13a to 13c show another embodiment of the drainage device according to the invention and of its covering. Here, the covering has a plate-shaped base element 12 whose dimensions, like those of the blank according to the above examples, can be chosen freely and adapted to the wound. Subcomponents in the form of longitudinal and transverse strips 13a, 13b are now arranged on the base element 12 and fixedly connected to the latter at least in some areas. The longitudinal and transverse strips 13a, 13b extend approximately perpendicular to one another. The base element 12 does not have a shape memory, but these strips 13a, 13b do. The base element is preferably made from a fluid-impermeable polymer film and the strips 13a, 13b are again made from a shape memory polymer or a shape memory metal.

The strips 13a, 13b preferably act as contraction tapes when they have reached the corresponding temperature that activates the shape memory. Here too, this temperature is preferably human body temperature, i.e. about 37° C. The strips 13a, 13b are adapted in the usual way to the size of the wound. The base element 12 and tapes 13a, 13b can be supplied as one unit and can be together cut to the desired size. However, they are preferably separate parts that are first adapted to the corresponding wound and then joined together.

As can be seen from the arrows in FIG. 13b, the strips 13a, 13b change shape as the temperature changes and, for example, adopt an undulating, shortened shape. In this way, the base element 12 lying underneath them also changes shape, in particular being drawn together. This also leads to a contraction of the wound and to a constant traction or pressure on the wound margins. The acting forces F are also shown again in FIG. 13c.

As can be seen from this figure, the cavity C in this embodiment is preferably filled at least partially with a wound bed filler F, for example a textile. A wound bed filler of this kind is not needed in the other embodiments and is also only optional here. It can, however, be used also in the other embodiments. Here too, the abovementioned sensors and sources can again be used. Application of a vacuum is also possible.

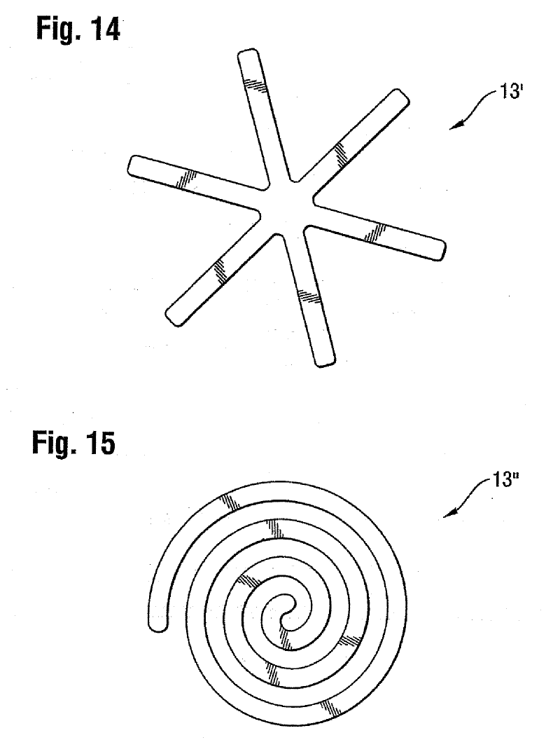

Instead of the strips 13a, 13b, it is also possible to use star-shaped subcomponents 13′ (see FIG. 14), spiral-shaped subcomponents 13″ (see FIG. 15) or other shapes produced from shape-memory materials.

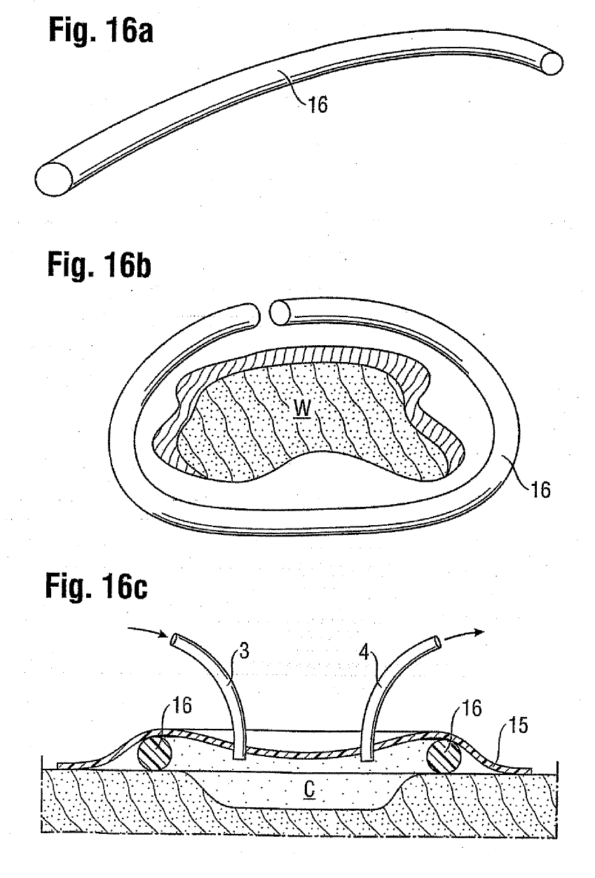

FIGS. 16a to 16c show another embodiment of the device according to the invention. Here, a bendable profile element or cord 16 is present which, in the normal state, is rectilinear or slightly curved, as can be seen from FIG. 16a. It can be applied in a ring shape around the wound W, as is shown in FIG. 16b. It is held in its position by means of a cover film or adhesive sheeting or film 15, which is placed over the internal circle defined by the profile element 16, over the profile element 16 itself and onto the healthy skin outside the cord 16 and is affixed there to the skin. Instead of a pressure-sensitive adhesive film, the film can also be affixed to the margins by an adhesive tape. However, it preferably also adheres on the profile element 16. The film 15 can be provided with through-openings for the tubes 3, 4, or it can simply be pierced in order to create these openings.

When there is a negative pressure in the cavity C, a force is applied to the film 15 by the atmospheric external pressure. The force results in a perpendicular force acting on the ring-shaped profile element 16, and this causes a contraction of the wound. It is thus possible, even without using a sponge in the wound bed, to avoid the film 15 attaching itself to the wound.

Moreover, the profile height of the ring 16 preferably increases during use. The profile element 16 can in this case have a shape memory such that, on reaching human body temperature, it increases its diameter and thus tensions the film more and exerts a traction on the wound margins. However, it can also have a relatively high coefficient of thermal expansion. For this purpose, it is applied to the wound and connected to the film 15 preferably at a relatively low temperature, after which it expands when heated and tensions the film.

The increase in size of the profile can also be achieved by mechanical means, for example by a spring incorporated in the profile element, or by inflating the profile element.

Possible materials for the cord are a polymer with shape memory, for example block copolymers. Possible materials for the film 15 are polymers. The film preferably has a thickness of 0.1 to 2 mm. The profile element 16 preferably has a diameter of ca. 5 to 30 mm. The profile element is preferably a solid profile.

It will be appreciated that the abovementioned elements are impermeable to air and liquid if the intention is to apply a negative pressure. Likewise, through-openings are to be suitably closed off in a manner impermeable to air and liquid.

### LIST OF REFERENCE SIGNS

1 cover plate
10 first through-opening
10′ second through-opening
11 blank for cover plate
12 base element
13a longitudinal strip
13b transverse strip
13′ star-shaped subcomponent
13″ spiral-shaped subcomponent
14 adhesive film
15 cover film and adhesive film
16 profile element
2 adhesive film
3 delivery tube
4 drainage tube
5 hydrogel
6 ultrasound transmitter/light source
60 electrical line
61 sound waves/light
7 temperature sensor/hydrosensor
70 signal line
8 flow sensor
80 signal line
9 heating element
A healthy tissue
W wound
C cavity
V vein
F filler for wound bed

1. A device for treating wounds of a patient, wherein the device comprises a covering for forming a low-pressure chamber over the wound, wherein the covering can be secured on the skin surrounding the wound, characterized in that, after the covering has been applied to the wound and secured on the skin, the shape of the covering can change from a basic shape to a predefined shape for use, as a result of which a contraction of the wound takes place, and in that the covering can change automatically from the basic shape to the shape for use.

2. The device as claimed in claim 1, wherein the covering is made at least partially from a material with shape memory.

3. The device as claimed in claim 2, wherein the covering is made entirely from a material with shape memory.

4. The device as claimed in claim 2, wherein the covering is made at least partially from a polymer with shape memory properties.

5. The device as claimed in claim 2, wherein the covering has the shape for use at a temperature that corresponds approximately to a body temperature of the patient.

6. The device as claimed in claim 2, wherein the covering has the basic shape at a temperature that corresponds approximately to room temperature.

7. The device as claimed in claim 2, wherein the covering comprises a base element without shape memory and at least one subcomponent with shape memory that is arranged on or in this base element, wherein the at least one subcomponent extends over the base element in such a way that, upon changing shape, it forces the base element to undergo a predetermined change of shape.

8. The device as claimed in claim 7, wherein the base element is an approximately plane plate, and the at least one subcomponent is a strip that extends over the plate.

9. The device as claimed in claim 8, wherein at least two strip-shaped subcomponents are present, which are arranged approximately at right angles to each other.

10. The device as claimed in claim 8, wherein the strip extends in a spiral shape over the plate.

11. The device as claimed in claim 7, wherein the base element is an approximately plane plate, and the at least one subcomponent is star-shaped.

12. The device as claimed in claim 1, wherein the covering is provided with at least one mechanical element by means of which its shape can be changed.

13. The device as claimed in claim 12, wherein the mechanical element is at least one restoring spring.

14. The device as claimed in claim 1, wherein the covering in the basic shape has an approximately plane configuration.

15. The device as claimed in claim 1, wherein the covering in the shape for use has an approximately dome-shaped configuration in which the dome curves away from the wound.

16. The device as claimed in claim 1, wherein the covering has at least two through-openings for the passage of delivery and drainage tubes.

17. The device as claimed in claim 1, wherein the covering comprises a profile element, which can be bent to form a ring, and a cover film that can be stretched over the ring.

18. The device as claimed in claim 17, wherein the profile element has a shape memory.

19. The device as claimed in claim 17, wherein the cover film is made from a polymer.

20. The device as claimed in claim 17, wherein the cover film has at least two holes for the passage of delivery and drainage tubes.

21. The device as claimed in claim 1, wherein one or more of the following means are arranged on or in the covering: an ultrasound transmitter, a light source, a temperature sensor, a sensor for measuring the air humidity, a sensor for measuring the flow of blood, a sensor for measuring the bacterial colonization, a heating means.

22. A device for treating wounds of a patient, wherein the device comprises a covering for forming a low-pressure chamber over the wound, wherein the covering can be secured on the skin surrounding the wound, characterized in that the covering comprises a profile element, which can be bent to form a ring, and a cover film that can be stretched over the ring, wherein the cover film can be secured on the skin, and wherein at least one delivery tube and at least one drainage tube can be inserted through the cover film into a cavity formed by the covering, and wherein the height of the profile element can be changed in a controlled manner during use.

23. A system for treating wounds, with a device for treating wounds as claimed in claim 1 and with a vacuum pump that can be connected to said device and is used to generate a low pressure in a cavity generated by the device for treating wounds.

24. A method for treating wounds, wherein a covering is secured on the wound, wherein the covering has a basic shape when being secured, and wherein the covering, after it has been secured in place, is changed to a shape for use.
